(19) United States
(12) Patent Application Publication
(10) Pub. No.: US 2007/0237349 A1
(43) Pub. Date: Oct. 11, 2007

(54) Earbud earphone and cushion therefor

(76) Inventors: Mark Donaldson, Auckland (NZ); Jonathan David Lowy, Auckland (NZ)

Correspondence Address: Daniel D. Chapman;Jackson Walker, LLP, Suite 2100, 112 E. Pecan, San Antonio, TX, 78205, US

(21) Appl. No.: 11/391,114

(22) Filed: Mar. 28, 2006

(51) Int. Cl. H04R 25/00; A61B 7/02

(52) U.S. Cl. 381/380; 181/135

(57) ABSTRACT

An ear bud earphone for use with a feedback noise cancellation system has a speaker and a microphone positioned anteriorly of the speaker. A cushion is provided exteriorly of a housing of the earphone. The cushion has a first region having a first acoustic transmission property, and a second region having a second acoustic transmission property comprising a greater impedance to transmission of audible sound than the first acoustic property. In one embodiment the first region is provided for contact with an inner ear surface, and the second region is provided between the first region and the housing. The cushion can be provided as a separate article.

### FIELD OF INVENTION

This invention relates to ear bud earphones (i.e. earphone apparatus having a sound transducer adapted for location directly in or adjacent to the ear canal) and to cushions for such earphones. The invention also has particular application to ear bud earphones used in conjunction with, or as part of, a noise cancellation system.

### BACKGROUND

Noise cancellation headphones provide the wearer with an ability to listen to sound free from the disturbing effects of background noise. Noise cancellation headphones are used widely in commercial passenger, and general, aviation and are now experiencing adoption in the mainstream in a variety of consumer audio applications.

Headphones, whether passive or noise cancellation, can be designed in either a supra aural or circum aural configuration. In the case of the former, the headphone rests on top of the ear with the interface to the wearer typically being soft open-cell foam. In the case of the latter, the ear cup completely encloses the ear with the human-headphone interface typically being a foam based leatherette ear pad.

Noise cancellation headphones are also configured in circum aural or supra aural arrangements. Circum aural noise cancellation headphones, however, tend to provide a better overall noise suppression effect, as the complete seal provided by the ear pad insulates the ear from the higher frequencies of sound which are more difficult to reduce by active noise cancellation techniques.

Headphones, whether passive or noise cancellation, are typically large and comprise a headband that can either be worn on top of the head or behind the neck. Headphones can be clumsy, uncomfortable and space consuming, especially for those who travel frequently.

An alternative solution for the personal reproduction of sound is the ear bud earphone (ear bud). The ear bud comprises one or two small audio transducers that are placed directly in or adjacent to the ear canal. Ear buds are used widely with hands-free cellular phone kits and portable audio devices such as mp3 and DVD players.

Presently few noise cancellation ear buds solutions exist in the marketplace. The few products that have been developed and commercialised tend to rely on a feed forward active noise cancellation configuration.

A feed forward active noise cancellation system is relatively simple in that it relies on a reference signal to generate a control response; this reference signal being some how related to the signal requiring control.

In the case of an ear bud solution the best choice of reference signal is a measure of the ambient noise directly outside of the ear bud's seal against the ear. This reference signal, obtained by way of a microphone transducer, is processed by noise cancellation electronic circuitry (filters) to generate an appropriate control response. The circuitry is designed to replicate the dynamic behaviour of the acoustic system between the reference measurement and control positions. All things being equal the control response, once output via the ear bud's speaker will effect cancellation of the noise that has infiltrated the ear canal. A feed forward controller is ‘dumb’ in the sense that it does not have any measure of its own performance. It relies on a prior knowledge of the disturbance (noise) and the acoustic system.

Unfortunately, in the case of feed forward control for noise cancellation systems used with ear buds, the reference signal is not fully representative of the noise that actually penetrates the ear bud's seal and enters the ear canal. The maximum performance of a feed forward active noise cancellation system can be calculated mathematically by measuring the coherence between the reference signal and the sound that penetrates the ear canal. This can be significantly less than unity, especially where the ear bud does not present a tight seal around the ear canal or the acoustics of the ear canal varies from that measured to determine the control filters.

A feedback control configuration relies on an error measurement located downstream from the point of control. The error represents a logical difference between a desired outcome and the measured result. However, as the control response of a feedback control configuration is directly related to its own output it is far more susceptible to an instability condition. This is especially true where the system under control is subject to change. In the context of active noise cancellation instability manifests itself as an uncontrolled ringing. Such a condition is problematic as it is unpleasant and can damage the hearing organ.

Ear bud earphones present a very different acoustic environment from traditional headsets. Therefore, providing an ear bud earphone for use with a feedback noise cancellation control system presents instability and performance problems.

### SUMMARY OF THE INVENTION

It is an object of the present invention to provide an earphone or earphone cushion which will allow the limitations of a feed forward noise cancellation ear bud earphone to be overcome by applying feedback control methods with an acoustic configuration conducive for such an application.

Alternatively or additionally it is an object of the present invention to provide an effective means of providing active noise cancellation in an ear bud that is also stable and robust.

Alternatively or additionally it is an object of the invention to provide an improved ear bud earphone, or an improved ear bud earphone cushion, or an improved earphone noise cancellation system, or to at least provide the public with a useful alternative.

Accordingly in one aspect the invention broadly consists in an ear bud earphone having a housing including a speaker and a microphone, and a cushion provided exteriorly of the housing, the cushion having a first region having a first acoustic transmission property, and a second region having a second acoustic transmission property, the second acoustic property including greater impedance to transmission of audible sound than the first acoustic property.

Preferably the first region is provided for contact with an inner ear surface, and the second region is provided between the first region and the housing.

Preferably the speaker is supported by the housing, and the microphone is supported by the housing and located anteriorly of the speaker to provide a signal for use by a noise cancellation system.

Preferably the microphone is provided on or adjacent to a side of the housing, so as to be provided adjacent to a wall of the ear canal in use.

Preferably a leaky acoustic path is provided between a user's ear canal and the ambient external environment.

Preferably the cushion provides the leaky acoustic path. The cushion may include an open cell foam material.

In a further aspect the invention provides a cushion for an ear bud earphone, the cushion having a first region for contact with an inner ear surface, the first region having a first acoustic transmission property, and a second region having a second acoustic transmission property, the second region being provided for location between the first region and a housing of the earphone.

Preferably the second acoustic property includes a greater impedance to transmission of audible sound than the first acoustic property.

In a further aspect the invention provides a method of forming a cushion for an ear bud earphone, the method including the steps of providing a sheet material selected from a foam material or a fibrous material, applying a composition to a selected region of the material, the composition resulting in an increased acoustic impedance of the selected region, and forming the material into a cushion dimensioned to fit about an ear bud earphone.

In a further aspect the invention provides an ear bud earphone including a housing, a speaker supported by the housing to produce sound, and a microphone supported by the housing and located anteriorly of the speaker to provide a signal for use by a noise cancellation system.

Preferably the microphone is provided on or adjacent to a side of the housing, so as to be provided adjacent to a wall of the ear canal in use.

Preferably a leaky acoustic path is provided between a user's ear canal and the ambient external environment. In one embodiment a cushion provides the leaky acoustic path.

In a further aspect the invention provides an ear bud earphone feedback noise cancellation system including an ear bud earphone having a speaker and a microphone and feedback noise cancellation circuitry adapted to receive a feedback signal from the microphone and provide a signal to the speaker to in use provide noise suppression.

In a further aspect the invention provides an ear bud earphone having a housing including a speaker and a microphone, and a cushion provided exteriorly of the housing, the cushion having a first region having a first acoustic transmission property, and a second region having a second acoustic transmission property, the second acoustic property including greater impedance to transmission of audible sound than the first acoustic property, the first region being provided for contact with an inner ear surface, and the second region being provided between the first region and the housing.

In a further aspect the invention provides a cushion for an ear bud earphone, the cushion having a first region for contact with an inner ear surface, the first region having a first acoustic transmission property, and a second region having a second acoustic transmission property, the second region being provided for location between the first region and a housing of the earphone, and wherein the second acoustic property includes a greater impedance to transmission of audible sound than the first acoustic property.

Further aspects of the invention will become apparent from the following description.

### DRAWING DESCRIPTION

The invention will be described by way of example with reference to:

FIG. 1 which is a diagrammatic cross section through a feedback controlled noise cancellation ear bud earphone,

FIG. 1A which is a diagram of an example of a feedback control system using an ear bud earphone according to the invention,

FIG. 1B which is a cross section through an other embodiment of a feedback controlled noise cancellation ear bud earphone,

FIG. 2 which is a cushion for an ear bud earphone, and

FIG. 3 which is a diagrammatic cross section through the cushion of FIG. 2.

### DETAILED DESCRIPTION

As discussed above, providing a headphone with a complete seal between a user's ear and the ambient external environment is an approach that has been used to improve noise cancellation performance, since this insulates the ear from high frequency sound that is difficult to suppress using noise cancellation systems.

However, we have found that providing a tight seal between an ear bud and the inner ear surfaces poses two problems. Firstly, a tight seal typically causes discomfort to the user because the pressure on parts of the inner ear. Secondly, a tight seal can lead to stability problems when feedback control is implemented. We have found that improved performance of an ear bud using a feedback noise cancellation system can be obtained if the seal between the ear bud and the surfaces of the inner ear is slightly acoustically “leaky” so that a leaky path is provided between the ear canal and the ambient environment. This is because such a path can provide both high frequency damping to preferably improve gain margin, and control over the acoustic system's open-loop sensitivity, especially when the ear bud is inserted into the ear canal.

Conventional ear bud earphones are supplied with a foam cushion which is applied about the earphone body. The cushion separates the earphone body from the user's ear and has the primary purpose of allowing a comfortable fit between inner surfaces of the ear and the earphone body. When used with an ear bud earphone, such cushions may also be referred to as “covers” or “boots”.

The foam is usually open cell foam which is comfortable for the user and allows the sound from the drive unit provided in the earphone body to pass through to the ear generally unimpeded.

A difficulty with the comfortable open cell foams is that the leakage path between the ear and the earphone body is too great, causing diminished performance.

One way to improve performance is to reduce the leakage through the leakage path. However, this typically reduces the comfort experienced by the user.

In one embodiment the invention may comprise two parts, noise cancellation apparatus in the form of a noise cancellation controller, and an ear bud earphone (having one or more “earbuds”). The invention can be configured to have one or two ear buds, each connecting to the noise cancellation controller. The noise cancellation controller can also be designed to accept voice signals as may be found on a cellular phone hands free system.

The noise cancellation controller comprises a printed circuit board (PCB) with various discrete components, or preferably an integrated circuit (IC), which embodies a control law (described further below) designed to achieve effective noise suppression when connected to the ear bud.

The ear bud comprises two transducers—a speaker and a microphone. The microphone is positioned anterior to the speaker and as close to the ear canal as practicable.

A special configuration of transducers, foams and seals, is provided in embodiments of the invention which act together to enable the device to provide effective and stable performance when connected to the noise cancellation electronics.

In another embodiment the invention relates to a cushion for an ear bud earphone, or to an ear bud earphone including such a cushion.

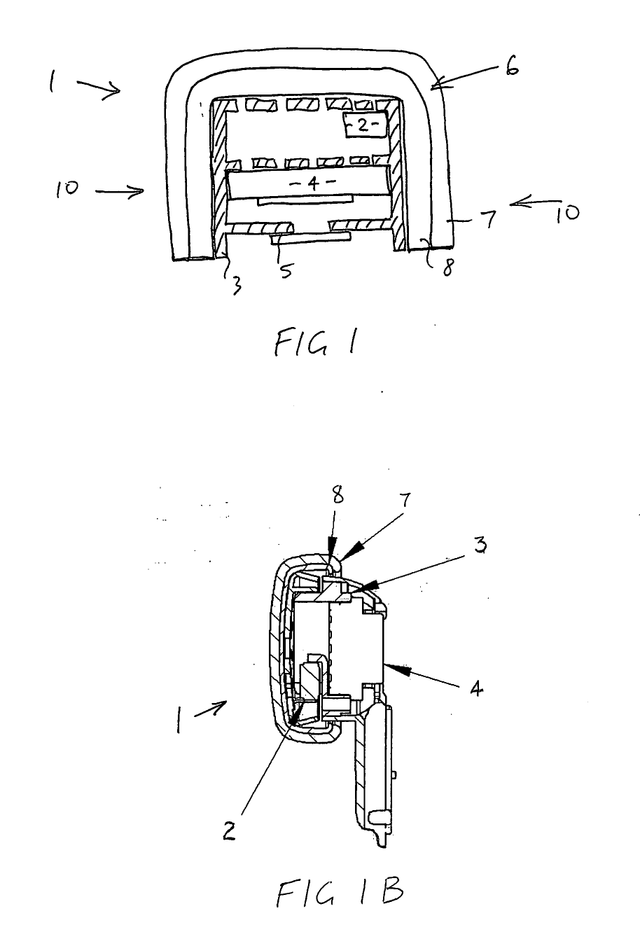

As shown in FIG. 1, an ear bud earphone I according to one aspect of the invention includes a microphone (or similar sound transducer) 2 which is supported by a housing structure 3 and is positioned in front of a speaker (or similar sound transducer) 4. In one embodiment a baffle plate 5 which may comprise part of the housing ensures that there is no direct, unfiltered leakage of sound from the front side of the baffle plate to the rear side. Leakage of sound is possible but only through cushion 6 (which may also provide part of the housing). In another embodiment a rear vent is provided behind the speaker to allow a controlled acoustic path from the rear of the speaker to the ambient external environment.

FIG. 1A illustrates the various control parameters of the control system—r(s) being the reference signal (for example an audio signal), e(s) being the error signal from the microphone 2, u(s) being the signal provided by an electronic controller (represented by function C(s)), y(s) being the output of the plant (i.e. the speaker 4 and acoustic path which are represented by G(s)), and w(s) being an acoustic disturbance.

The control law is given by:
e(s)=1/(1+C(s)G(s))

Thus the larger the gain, the lower the error. Hence, damping of higher frequencies at which instability may occur is desirable as higher gain may be used.

Cushion 6 is preferably constructed from a foam material, however other materials may be used such as fibrous materials. The most preferred foam is a high density open cell foam, although other foams such as cellular, reticulated, or memory foams could be used. The cushion provides a leaky path for the sound produced by the speaker to travel to ambient conditions. This is desirable since we have found that this path may simulate the effect of ear pads on a circum aural headphone providing both high frequency damping to preferably improve gain margin and control over the acoustic system's open-loop sensitivity, especially when inserted into the ear canal.

As can be seen from FIG. 1, the cushion has two regions 7 and 8 which have different acoustic transmission properties. In one embodiment region 7 comprises high density open cell foam which passes sound substantially unimpeded. This material also has a “soft” feel, which is comfortable against inner ear surfaces of a user.

In the embodiment illustrated, region 8 is provided between region 7 and the housing 3. Region 8 has a different acoustic property to region 7 in that it can significantly impede sound transmission. Sound from the speaker can still pass through the part of region 8 that is in front of the speaker, but those parts of region 8 that are located about the edges of the housing are compressed in use as shown by arrows 10 when the ear bud is located in the ear canal. Compression of this region significantly prevents passage of sound from the speaker to the ambient environment externally of the user's ear. However, an acoustically leaky path to the ambient environment is still provided.

Region 8 may be provided about sides only of the ear bud housing. Furthermore, the regions may blend into each other, rather than being distinctly separated as shown in the drawings.

In another embodiment, the position of the regions 7 and 8 may be reversed so that region 7 may be provided adjacent to the housing 3 and region 8 may be provided exteriorly of region 7. Furthermore, in a further embodiment the cushion may be provided such that region 7 is arranged as shown in the drawing figures and region 8 does not cover the anterior portion of the earphone i.e. so that region 8 is provided only about the sides of the earphone.

FIG. 1B shows another embodiment in which features that are the same as or similar to those described with reference to FIG. 1 have like reference numerals.

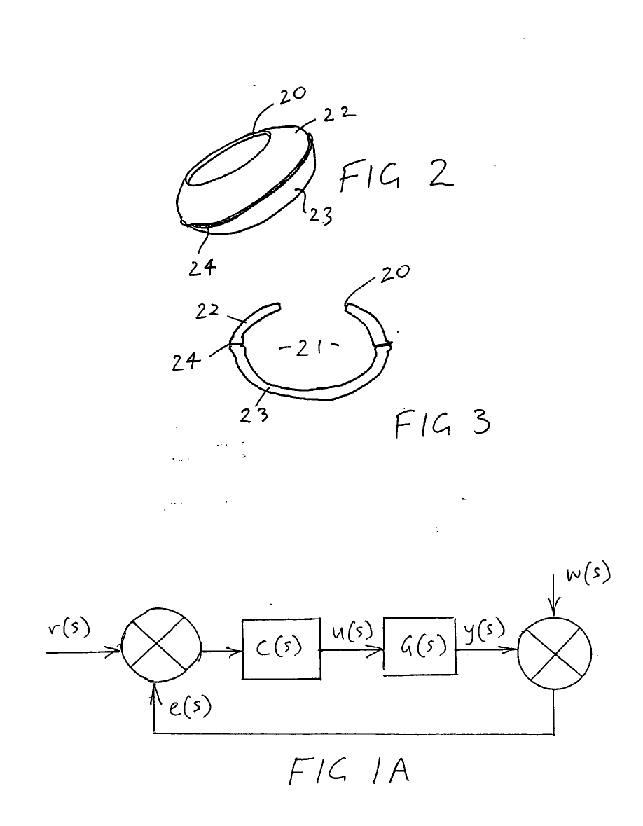

The cushion may be provided as a component which is separable from the housing, as shown in FIGS. 2 and 3 in which the cushion can be seen having an entrance 20 to an interior cavity 21 within which the ear bud housing can be disposed in use.

In a preferred embodiment the cushion is constructed from a sheet of high density open cell foam material which is coated on one side with a composition, preferably a flocking agent such as a paint or similar material which when dry provides region 8 having the increased acoustic attenuation characteristic described above. The increased acoustic attenuation is caused by some or all of the cell formations in the foam or fibrous material being closed or partially closed by the flocking agent.

The sheet material is then cut (preferably by a punch process) to provide an upper side 22 with entrance aperture 20 and a lower side 23. The two sides are joined about joint 24 (for example using a suitable glue). To provide a cushion such as that shown in FIG. 1, the sides are joined with region 8 innermost.

The cushion 6 protects the wearer from the high frequency noise that is not cancelled by active means. The untreated region 7 of closed cell foam provides a more comfortable and accurate fit to the wearer's unique in-ear topography while the inner region 8 allows controlled attenuation of sound transmission to the ambient environment. As referred to above, other materials may be used, for example other foam types, and the location of the regions may be exchanged. In another embodiment, a fibrous or woven material may be provided which can be coated on one side with a flocking agent to create a region in which sound transmission is impeded. In another embodiment a fibrous or woven material may be manufactured with differing densities to create regions having different acoustic transmission properties to enable the desired acoustic performance to be achieved.

In a preferred embodiment a high force factor speaker 4 is employed. The dynamics of this speaker can be controlled by sealing it in a semi-closed cavity with the only exit being a vent covered by a filter membrane (as described above), which only passes the lower frequencies of sound.

In a preferred embodiment the microphone 2 is of the electret condenser (ECM) variety, and exhibits a flat frequency response across the bandwidth of 20 Hz to 10 kHz with a high signal to noise ratio.

The controller implements a control law described above such that embodiments of the invention provide noise cancellation up to 20 dB peak over a bandwidth of 1000 Hz.

1. An ear bud earphone comprising a housing having a speaker and a microphone, and a cushion provided exteriorly of the housing, the cushion having a first region having a first acoustic transmission property, and a second region having a second acoustic transmission property, wherein the second acoustic property comprises greater impedance to transmission of audible sound than the first acoustic property.

2. An ear bud as claimed in claim 1 wherein the first region is provided for contact with an inner ear surface, and the second region is provided between the first region and the housing.

3. An ear bud as claimed in claim 1 wherein the speaker is supported by the housing, and the microphone is supported by the housing and located anteriorly of the speaker to provide a signal for use by a noise cancellation system.

4. An ear bud as claimed in claim 1 wherein the microphone is provided on or adjacent to a side of the housing, so as to be provided adjacent to a wall of the ear canal in use.

5. An ear bud as claimed in claim 1 wherein a leaky acoustic path is provided between a user's ear canal and the ambient external environment.

6. An ear bud as claimed in claim 5 wherein the cushion provides the leaky acoustic path.

7. An ear bud as claimed in claim 6 wherein the cushion includes an open cell foam material.

8. An ear bud as claimed in claim 7 wherein the foam material in use contacts a user's ear canal.

9. An ear bud as claimed in claim 1 wherein the first region substantially covers an anterior region of the earphone.

10. An ear bud as claimed in claim 9 wherein the second region does not cover the anterior region of the earphone.

11. A cushion for an ear bud earphone, the cushion comprising a first region for contact with an inner ear surface, the first region having a first acoustic transmission property, and a second region having a second acoustic transmission property, the second region being provided for location between the first region and a housing of the earphone.

12. A cushion as claimed in claim 11 wherein the second acoustic property includes a greater impedance to transmission of audible sound than the first acoustic property.

13. A method of forming a cushion for an ear bud earphone, the method comprising the steps of providing a sheet material selected from a foam material or a fibrous material, applying a composition to a selected region of the material, the composition resulting in an increased acoustic impedance of the selected region, and forming the material into a cushion dimensioned to fit about an ear bud earphone.

14. An ear bud earphone comprising a housing, a speaker supported by the housing to produce sound, and a microphone supported by the housing and located anteriorly of the speaker to provide a signal for use by a noise cancellation system.

15. An ear bud as claimed in claim 14 wherein the microphone is provided on or adjacent to a side of the housing, so as to be provided adjacent to a wall of the ear canal in use.

16. An ear bud as claimed in claim 14 wherein a leaky acoustic path is provided between a user's ear canal and the ambient external environment.

17. An ear bud as claimed in claim 16 wherein a cushion provides the leaky acoustic path.

18. An ear bud as claimed in claim 17 wherein the cushion includes an open cell foam material.

19. An ear bud as claimed in claim 17 wherein the foam material in use contacts a user's ear canal.

20. An earbud earphone feedback noise cancellation system comprising an ear bud earphone having a speaker and a microphone and feedback noise cancellation circuitry adapted to receive a feedback signal from the microphone and provide a signal to the speaker to in use provide noise suppression.

21. An ear bud earphone comprising a housing including a speaker and a microphone, and a cushion provided exteriorly of the housing, the cushion having a first region having a first acoustic transmission property, and a second region having a second acoustic transmission property, the second acoustic property including greater impedance to transmission of audible sound than the first acoustic property, the first region being provided for contact with an inner ear surface, and the second region being provided between the first region and the housing.

22. A cushion for an ear bud earphone, the cushion comprising: a first region for contact with an inner ear surface, the first region having a first acoustic transmission property, and a second region having a second acoustic transmission property, the second region being provided for location between the first region and a housing of the earphone, and wherein the second acoustic property comprises a greater impedance to transmission of audible sound than the first acoustic property.
